SEQUENCE LISTING (34 sequences)

SEQ ID NO: 1 - DNA, 66 bp, Artificial
  Donor Oligonucleotide for targeted nucleotide exchange
ttccaagctcttagctatcactaattttuuagugauagctaagagcuuggaagcgcgttttcgcgc

SEQ ID NO: 2 - DNA, 80 bp, artificial
  Donor Oligonucleotide for targeted nucleotide exchange
atgttaagttgtatcctccgctctccagcttagtgatacctaagagcttccaagacaacttaactttgctacctaatctt

SEQ ID NO: 3 - DNA, 68 bp, artificial
  Donor Oligonucleotide for targeted nucleotide exchange
cgctgttgaacatgattggcgggtcttttgacccgccaatcatguucaacagcagcgcgttttcgcgc

SEQ ID NO: 4 - DNA, 71 bp, artificial
  Donor Oligonucleotide for targeted nucleotide exchange
gacttggacagaattccggtgtttgtagacccgccaatcatgttcaacagcaaaattcttccgggcggtgc

SEQ ID NO: 5 - DNA, 20 bp, artificial
  primer
gcataataggtggtactttt

SEQ ID NO: 6 - DNA, 20 bp, artificial
  primer
gctgcaactggtgggtaata

SEQ ID NO: 7 - DNA, 20 bp, artificial
  primer
taatgacggtgccatctcat

SEQ ID NO: 8 - DNA, 20 bp, artificial
  primer
ctagaaactgaggaatggct

SEQ ID NO: 9 - DNA, 64 bp, artificial
  Donor Oligonucleotide for targeted nucleotide exchange
gttatgcaattggaagatcggttttttaaccgaucuuccaatugcauaacgcgcgttttcgcgc

SEQ ID NO: 10 - DNA, 80 bp, artificial
  Donor Oligonucleotide for targeted nucleotide exchange
aatgtgtgagctcggttagctttgtagaaccgatcttccaattgcataaccatgccaagatgctggttgtttaataaaag

SEQ ID NO: 11 - DNA, 68 bp, artificial
  Donor Oligonucleotide for targeted nucleotide exchange
caggacaagtccagccgtcgtatgattttucauacgacggctggacuuguccuggcgcgttttcgcgc

SEQ ID NO: 12 - DNA, 79 bp, artificial
  Donor Oligonucleotide for targeted nucleotide exchange
cggagtctcttgaaacgcatctgtaccaatcatacgacgctggacttgtcctgtgattgctacaagaggaacactatct

SEQ ID NO: 13 - DNA, 80 bp, artificial
  Donor Oligonucleotide for targeted nucleotide exchange
aagttgtatcctccgctctccagcttagtgatacctaagagcttccaagacaacttaactttgctacctaatcttgcaga

SEQ ID NO: 14 - DNA, 80 bp, artificial
  Donor Oligonucleotide for targeted nucleotide exchange
gtatgtcaagtgatatcctcctttttctgacttagtaatgcttaaaagcttccatgatagttttaatttgcttcccaatc

SEQ ID NO: 15 - DNA, 80 bp, artificial
  Donor Oligonucleotide for targeted nucleotide exchange
cgtcttagacaaaatttcaggattttttgctcctccaatcatgttcaagagtagcacccgaccttttggggcacggttag

SEQ ID NO: 16 - DNA, 79 bp, artificial
  Donor Oligonucleotide for targeted nucleotide exchange
caacaataggagtttcctgaaaagcatcagtacctatcatcctacgttgcacttgacctgttatagcaacaatggggac

SEQ ID NO: 17 - DNA, 80 bp, artificial
  Donor Oligonucleotide for targeted nucleotide exchange
ccccaggtatgtgtgtgctctgttagccttatagaaccgatcctccaattgaaccaccattcccaagtgttgattattca

SEQ ID NO: 18 - DNA, 68 bp, artificial
  Donor Oligonucleotide for targeted nucleotide exchange
atggaagcttttaagcattactaagttttcuuaguaaugcttaaaagcuuccaugcgcgttttcgcgc

SEQ ID NO: 19 - DNA, 66 bp, artificial
  Donor Oligonucleotide for targeted nucleotide exchange
actcttgaacatgattggaggagcttttgcuccuccaatcatguucaagagugcgcgttttcgcgc

SEQ ID NO: 20 - DNA, 68 bp, artificial
  Donor Oligonucleotide for targeted nucleotide exchange
caggtcaagtgcaacgtaggatgatttttaucauccuacgttgcacuugaccuggcgcgttttcgcgc

SEQ ID NO: 21 - DNA, 68 bp, artificial
  Donor Oligonucleotide for targeted nucleotide exchange
tggtggttcaattggaggatcggttttttaaccgauccuccaatugaaccaccagcgcgttttcgcgc

SEQ ID NO: 22 - DNA, 20 bp, artificial
  primer
catgaggaatcagttgagca

SEQ ID NO: 23 - DNA, 20 bp, artificial
  primer
tttgaaagtgcatctgctgc

SEQ ID NO: 24 - DNA, 25 bp, artificial
  primer
actaagtcagaaaaaggaggatatc

SEQ ID NO: 25 - DNA, 20 bp, artificial
  primer
aagaggatcttgagctttgg

SEQ ID NO: 26 - DNA, 20 bp, artificial
  primer
ggtcaagtgccacgtaggat

SEQ ID NO: 27 - DNA, 20 bp, artificial
  primer
gggtgcttcactttctgctc

SEQ ID NO: 28 - DNA, 20 bp, artificial
  primer
cccgtggcaagtactttgat

SEQ ID NO: 29 - DNA, 20 bp, artificial
  primer
ggattccccaggtatgtgtg

SEQ ID NO: 30 - DNA, 60 bp, artificial
  Donor Oligonucleotide for targeted nucleotide exchange
gacagccggaacacggcggcatcagagcagccgattgtctgttgtgcccagtcatagccg

SEQ ID NO: 31 - DNA, 68 bp, artificial
  Donor Oligonucleotide for targeted nucleotide exchange
agacaatcggctgctctgatgccgcttttgcggcaucagagcagccgauugucugcgcgttttcgcgc

SEQ ID NO: 32 - DNA, 80 bp, artificial
  Donor Oligonucleotide for targeted nucleotide exchange
gcccatccacactaggcaaggagaacttatgtctataatccactgattgttccttgaggagaaagaggatgagggaaagt

SEQ ID NO: 33 - DNA, 20 bp, artificial
  primer
cagtgtggcttgagtagtca

SEQ ID NO: 34 - DNA, 20 bp, artificial
  primer
tagatccagctggttccaaa

(19) United States
(12) Patent Application Publication
(10) Pub. No.: US 2009/0064377 A1
(43) Pub. Date: Mar. 5, 2009

(54) METHOD AND MEANS FOR TARGETED NUCLEOTIDE EXCHANGE

(76) Inventors: Daphne Yvette Rainey-Wittich, Ede (NL); Michiel Theodoor Jan de Both, Wageningen (NL); Paul Bundock, Amsterdam (NL)

Correspondence Address: BROWDY AND NEIMARK, P.L.L.C.;624 NINTH STREET, NW, SUITE 300, WASHINGTON, DC, 20001-5303, US

(21) Appl. No.: 12/088,813

(22) PCT Filed: Sep. 29, 2005
(86) PCT No.: PCT/NL05/00706

§ 371 Date: Sep. 8, 2008

(51) Int. Cl. A01H 5/00; C07H 21/04; C12N 9/99; C12N 9/00; C12N 5/10

(52) U.S. Cl. 800/298; 536/24.2; 435/184; 435/183; 435/419

(57) ABSTRACT

The invention pertains to a method for targeted alteration of a duplex acceptor DNA sequence, comprising combining the duplex acceptor DNA sequence with a donor oligonucleotide, wherein the duplex acceptor DNA sequence contains a first DNA sequence and a second DNA sequence which is the complement of the first DNA sequence and wherein the donor oligonucleotide comprises a domain that comprises at least one mismatch with respect to the duplex acceptor DNA sequence to be altered, preferably with respect to the first DNA sequence, and wherein a section of the donor oligonucleotide is methylated to a higher degree than the second DNA sequence and/or wherein the second DNA is methylated to a lower degree than the corresponding section of the donor oligonucleotide, optionally in the presence of proteins that are capable of targeted nucleotide exchange and to oligonucleotides for use in the method.

### FIELD OF THE INVENTION

The present invention relates to a method for the specific and selective alteration of a nucleotide sequence at a specific site of the DNA in a target cell by the introduction into that cell of an oligonucleotide. The result is the targeted exchange of one or more nucleotides so that the sequence of the target DNA is converted to that of the oligonucleotide where they are different. More in particular, the invention relates to the targeted nucleotide exchange using modified oligonucleotides. The invention further relates to oligonucleotides and kits. The invention also relates to the application of the method.

### BACKGROUND OF THE INVENTION

Genetic modification is the process of deliberately creating changes in the genetic material of living cells with the purpose of modifying one or more genetically encoded biological properties of that cell, or of the organism of which the cell forms part or into which it can regenerate. These changes can take the form of deletion of parts of the genetic material, addition of exogenous genetic material, or changes in the existing nucleotide sequence of the genetic material. Methods for the genetic modification of eukaryotic organisms have been known for over 20 years, and have found widespread application in plant, human and animal cells and micro-organisms for improvements in the fields of agriculture, human health, food quality and environmental protection. The common methods of genetic modification consist of adding exogenous DNA fragments to the genome of a cell, which will then confer a new property to that cell or its organism over and above the properties encoded by already existing genes (including applications in which the expression of existing genes will thereby be suppressed). Although many such examples are effective in obtaining the desired properties, these methods are nevertheless not very precise, because there is no control over the genomic positions in which the exogenous DNA fragments are inserted (and hence over the ultimate levels of expression), and because the desired effect will have to manifest itself over the natural properties encoded by the original and well-balanced genome. On the contrary, methods of genetic modification that will result in the addition, deletion or conversion of nucleotides in predefined genomic loci will allow the precise modification so existing genes.

Currently, two methods are known for creating precise targeted genetic changes in eukaryotic cells: gene targeting through homologous recombination and oligonucleotide-directed targeted nucleotide exchange.

Methods based on homologous recombination exploit the principle that naturally occurring or pre-induced double-strand breaks in the genomic DNA will be repaired by the cell using any available template DNA fragment with some nucleotide sequence homology to the flanking regions adjacent to the break (Puchta, Plant Mol. Biol. 48: 173, 2002; J. Exp. Botany, 2005, 56, 1). By supplying to such cell donor DNA with the required sequence homologies, homologous recombination at either end of the break may result in a precise repair, whereby any artificially designed modifications in between the homology regions of the donor DNA will be incorporated in the existing loci. In eukaryotic cells, this precise break repair occurs at rather low frequencies in favour of a less precise repair mechanism, in which parts of the sequence may be lost and non-related DNA sequence may be incorporated.

Oligonucleotide-directed Targeted Nucleotide Exchange (TNE, sometimes ODTNE) is a different method, that is based on the delivery into the eukaryotic cell nucleus of synthetic oligonucleotides (molecules consisting of short stretches of nucleotide-like moieties that resemble DNA in their Watson-Crick basepairing properties, but may be chemically different from DNA) (Alexeev, and Yoon, Nature Biotechnol. 16: 1343, 1998; Rice, Nature Biotechnol. 19: 321, 2001; Kmiec, J. Clin. Invest. 112: 632, 2003). By deliberately designing a mismatch nucleotide in the homology sequence of the oligonucleotide, the mismatch nucleotide may be incorporated in the genomic DNA sequence. This method allows the conversion of single or at most a few nucleotides in existing loci, but may be applied to create stop codons in existing genes, resulting in a disruption of their function, or to create codon changes, resulting in genes encoding proteins with altered amino acid composition (protein engineering).

Targeted nucleotide exchange has been described in plant, animal and yeast cells. The first TNE reports utilized a so-called chimera wherein the oligonucleotides may be synthesized as RNA-DNA hybrid molecules (Beetham et al., PNAS 96: 8774, 1999; Kochevenko and Willmitzer, Plant Physiol. 132: 174, 2003) and that consisted of a self-complementary oligonucleotide that is designed to intercalate at the chromosomal target site. The chimera contains a mismatched nucleotide that forms the template for introducing the mutation at the chromosomal target. The first examples using chimeras came from human cells (see the review Rice et al. Nat. Biotech., 2001, 19: 321-326; Alexeev et al. Nature Biotech, 2000, 18, 43). The use of chimeras has also been successful in the plant species tobacco, rice, and maize (Beetham et al. 1999 Proc. Natl. Acad. Sci. USA 96: 8774-8778; Kochevenko et al. 2003 Plant Phys. 132: 174-184; Okuzaki et al. 2004 Plant Cell Rep. 22: 509-512; Zhu et al. PNAS 1999, 96, 8768). These studies relied upon the introduction of Point mutations that confer herbicide resistance. The most tractable system to study TNE has been the yeast Saccharomyces cerevisiae (Rice et al. 2001 Mol. Microbiol. 40: 857-868). The use of yeast mutants has identified several genes (RAD51, RAD52 and RAD54) that seem to play a key role in TNE.

Alternative methods of TNE utilize single stranded (ss) oligonucleotides to introduce specific chromosomal mutations. Thus far, ss oligonucleotides have only been tested in yeast and human cells (Liu et al. 2002 Nucl. Acids Res. 30: 2742-2750; review, Parekh-Olmedo et al. 2005 Gene Therapy 12, 639-646).

The efficiency of TNE is increased when human cells are blocked in the S phase (Brachman et al. 2005 DNA Rep. (Amst) 4: 445-457), suggesting that the DNA must have an open configuration for efficient TNE to occur. TNE using ss oligonucleotides has been proposed to occur via a replication mode of gene repair. In this scenario the ss oligonucleotide would anneal at a replication fork and would be assimilated into the daughter strand between the Okasaki fragments of the lagging strand. This results in a mismatch in one of the newly replicated DNA strands. The mutation in the es oligonucleotide could drive the conversion of the nucleotide in the parental strand, but the likelihood of this directional repair is probably low. This is because current views of mismatch repair during replication promote the notion that the parental strand is used as a template to repair errors in the daughter strand (Stojic et al. 2004 DNA Repair (Amst) 3: 1091-1101). TNE using chimeric oligonucleotides is thought to occur by intercalation of the chimera into the DNA duplex. The chimera RNA strand binds to one strand of the target sequence. This RNA/DNA binding is more stable than a DNA/DNA interaction and may help to stabilize the chimera in the duplex. The gene correction is carried out by the DNA strand of the chimera that binds to the other target strands resulting in a base change in one strand of the target sequence. After degradation or dissociation of the chimera the resulting mismatch in the target is probably resolved by proteins from the mismatch repair pathway, but this is as yet unconfirmed.

The greatest problem facing the application of TNE in cells of higher organisms such as plants is the low efficiency that has been reported so far. In maize Zhu et al. (2000 Nature Biotech. 18: 555-558) reported a conversion frequency of 1×10⁻⁴. Subsequent studies in tobacco (Kochevcnko et al. 2003 Plant Phys. 132: 174-184) and rice (Okuzaki et al. 2004 Plant Cell Rep 22: 509-512) have reported frequencies of 1×10⁻⁶ and 1×10⁻⁴ respectively. These frequencies remain too low for the practical application of TNE.

Faithful replication of DNA is one of the key criteria that mediates maintenance of genome stability and ensures that the genetic information contained in the DNA is passed on free of mutation from one generation to the next. Many errors arise from damage in the parental DNA strand or are generated by agents that react with DNA bases (UV light, environmental toxins). Every organism must maintain a safeguard to prevent or correct these mutations. The mismatch repair system (MMR) is thought to recognize and correct mismatched or unpaired bases caused during DNA replication, in DNA damage surveillance and in prevention of recombination between non-identical sequences (Fedier and Fink, 2004 Int J Oncol. 2004; 24(4):1039-47), and contributes to the fidelity of DNA replication in living cells.

MMR recognizes and eliminates misincorporated nucleotides on the newly synthesized strand by an excision/re-synthesis process during replication and thus restores the information contained on the template strand (Jiricny, Mutat Res., 1998, 409(3), 107-21; Kolodner and Marsischky, Mol Cell. 1999, 4(3), 439-444; J. Biol. Chem. 1999, 274(38), 26668-26682; Curr. Opin. Genet. Dev., 1999, 9(1), 89-96; Fedier and Fink, 2004 Int J Oncol. 2004; 24(4):1039-47). This type of replication error occurs spontaneously and is occasionally not detected by 3′-5′ exonuclease proofreading activity of the replicative DNA polymerase enzyme complex, or is caused by modified nucleotides in the template strand (Fedier and Fink, 2004 Int J Oncol. 2004; 24(4):1039-47). Mutations in human MMR proteins have been found to lead to the generation of microsatellite sequences (Fedier and Fink, 2004 Int J Oncol. 2004, 24(4):1039-47). Microsatellites are genetic loci of 1-5 base pair tandem repeats, repeated up to 30 times. These sequences can cause slippage of the DNA polymerase during replication, resulting in the formation of small loop heteroduplexes of one or more nucleotides in the template or nascent DNA strand. Failure to remove these heteroduplexes produces alleles of different sizes in each subsequent round of replication. These alleles have been shown to be present in cancer patients with defective MMR proteins (Peltomaki, J Clin Oncol. 2003, 21(6), 1174-9; Fedier and Fink, 2004 Int J Oncol. 2004, 24(4):1039-47, Jiricny and Nystrom-Lahtl, Curr Opin Genet Dev. 2000, 10(2), 157-61).

MMR is also involved in DNA damage signaling via linkage to cell cycle checkpoint activation and apoptosis initiation pathways in the presence of DNA damage (Bellacosa, J Cell Physiol. 2001, 187(2), 137-44.). MMR induces apoptosis to avoid the accumulation of a large number of mutations and thus the absence of the system has been examined in terms of interaction with anti-tumor agents. Patients with compromised MMR systems responded less effectively to treatment with antitumor agents in destruction of tumor cells, thus indicating the importance of the apoptotic induction of the MMR system in drug response (Aquilina and Bignami, J Cell Physiol. 2001 May; 187(2):145-541).

Thus MMR has a dual role in maintaining genomic stability: 1) recognition and correction of mismatches and 2) signaling apoptosis to prevent accumulation of mutations.

The MMR system of bacteria consists of three main proteins, which are referred to as the MutHLS proteins. MutS is an ATPase involved in mismatch recognition. It binds and hydrolyses ATP in the process of binding the mismatched base pairs (Baitinger et al., J Biol Chem. 2003 Dec. 5; 278(49):49505-11). ATP promotes release of MutS from the mismatch. MutL assists mismatch recognition by initiating and coordinating mismatch repair in the formation of a link between MutS and MutH. The N-terminal domain of this protein is the ATPase domain (Guarne et al., EMBO J. 2004 Oct. 27; 23(21):4134-45). Each domain of Mutt interacts with UvrD helicase to activate UvrD helicase activity and its ability to unwind double stranded DNA to a single strand form. The third protein of the system, MutH binds and nicks DNA with the same recognition sites as MboI and Sau3AI (Baitinger et al., J Biol Chem. 2003 Dec. 5; 278(49):49505-11; Giron-Monzon et al., Biol Chem. 2004 Nov. 19; 279(47):49338-45; Joseoh et al., DNA Repair (Amst). 2004 Dec. 2; 3(12):1561-77). Muth binds any DNA non-specifically in a co-operative and metal-dependent (Mg2+) manner (Baitinger et al., J Biol Chem. 2003 Dec. 5; 278(49):49505-11). The combination and interaction of MutL with ATP produces more specific binding of MutH to fully methylated DNA.

The addition of methyl group to the cyclic carbon to produce 5-methylcytosine increases the information provided by the ordered sequences of bases. In prokaryotes the methylation of DNA plays an important role in DNA repair and replication as well as in recognition and protection of self DNA. Cytosine-5-methylation is the most common DINA modification in plants and is essential for normal development, probably by ensuring the appropriate chromatin structure (Finnegan et al. 2000 Curr. Opin. Genet. Dev. 10: 217-223). Methylation of DNA occurs after DNA synthesis and is catalyzed by enzymes known as DNA methyltransferases. In Arabidopsis approximately 6% of all cytosine residues are methylated (Kakutani et al. 1999 Genetics 151: 831-838). The distribution of methylcytosine is not random, most methylated residues occur within repetitive DNA found in heterochromatin. However, methylcytosine is also found in single copy sequences where it is important in regulating gene expression (Jacobson & Meyerowitz 1997 Science 277: 1100-1103; Cubas et al. 1999 Nature 401: 157-161).

Methylcytosine can occur in any sequence context in plant DNA but it is most common in cytosines in sequences that are identical when read 5′ to 3′, the so-called symmetrical cytosines CpG and CpNpG. Symmetrical cytosines in such motifs are both methylated. Methylation patterns are transferred to the newly replicated daughter strands by maintenance methyltransferases, enzymes that preferably bind hemi-methylated substrates, and modify the unmethylated symmetric cytosines on newly synthesized DNA strands. Plants have 3 classes of cytosine methyltransferases. The first, the MET1 class of methyltransferase, is similar in structure to the mouse DNA de novo methyltransferase Dnmt1. This class consists of a small multigene family of five members (Genger et al. 1999 Plant Mol. Biol. 41: 269-278). The second family, the chromomethylases, contains at least 3 members in Arabidopsis (Genger et al. 1999 Plant Mol. Biol. 41: 269-278). Proteins of this family include and extra chromodomain which is important in targeting proteins to heterochromatin (Ingram et al. 1999 Plant Cell 11: 1047-1060) where they are thought to mediate methylation. The third family includes plant proteins showing homology to the mouse Dmnt3 methyltransferase. The role of this class in DNA methylation has yet to be identified although they are probably involved in methylating specific genomic regions.

The present inventors believe that the methylation status may serve to identify the parental strand of DNA. In bacteria, mismatch repair follows closely behind DNA replication. The parental DNA strand is methylated, but the newly synthesized strand is not, thereby allowing the mismatch repair machinery to recognize template (parental strand) from the newly synthesized strand which contains the mistaken base. MutH recognizes this adenine hemi-methylation in GATC sequences and cuts in the unmethylated (newly synthesized strand). Exonuclease removes approximately 20 bases of this strand and DNA polymerase synthesizes a new strand of DNA, which is then methylated at a later point.

The DNA mismatch repair genes are well conserved evolutionarily for prokaryotes and eukaryotes. Homologues of the bacterial MutL and MutS proteins have been found in all model eukaryotes (yeast, Arabidopsis, Drosophila, C. elegans, mouse and human). To date no MutH homolog has been found.

In mammals the relationship between DNA methylation is not as simple as in bacteria. Following DNA replication, mammalian DNA possesses a transient, strand-specific CpG hemi-methylation in the parental strand (Drummond and Bellacosa, Nucleic Acids Res. 2001 Jun. 1; 29(11):2234-43.). Maintenance cytosine methyltransferases then restore full methylation to hemi-methylated CpG sites.

As the efficiency of the current methods of ODTNE is relatively low (as stated previously; between 10⁻⁶ and 10⁻⁴, despite reported high delivery rates of the oligonucleotide of 90%) there is a need in the art to come to methods for TNE that are more efficient.

### DESCRIPTION OF THE INVENTION

The present inventors have now found that the use of demethylated genomic DNA in the cells to be used for TNE, optionally in combination with the use of synthetic oligonucleotides that (partly) comprise methylated nucleotides provides for an improved method for performing TNE.

Demethylation of genomic DNA can be achieved through chemical treatments of the biological material prior to DNA replication or through the use of methylation-deficient mutants. Both methods result in demethylated parental DNA strands. The use of demethylated genomic DNA alone, or the use of methylated synthetic oligonucleotides alone, or the combination of both, will result in a duplex or triplex DNA structure wherein the removal of the mismatched nucleotide in the parental strand, and the subsequent stable incorporation in the genome of the deliberate mismatched base designed in the oligonucleotide is achieved with high efficiency.

The present invention is also based on the inventive consideration that the desired targeted nucleotide exchange can be achieved by the use of (partly) methylated oligonucleotides. The methylation-status of the oligonucleotide can be varied as will be disclosed herein below.

The present invention thus, in one aspect provides ((fully) methylated) oligonucleotides. The oligonucleotides can be used to introduce specific genetic changes in plant and animal or human cells. The invention is applicable in the field of biomedical research, agriculture and to construct specifically mutated plants and animals, including humans. The invention is also applicable in the field of medicine and gene therapy.

The sequence of an oligonucleotide of the invention is homologous to the target strand except for the part that contains a mismatch base that introduces the base change in the target strand. The mismatched base is introduced into the target sequence. By manipulating the methylation of the nucleotides, and more in particular, by manipulating the degree of methylation of the oligonucleotide that introduces the mismatch and/or by manipulating the degree of methylation of one or both strands of the DNA duplex in which the oligonucleotide can intercalate, the efficiency (or the degree of successful incorporation of the desired nucleotide at the desired position in the DNA duplex) can be improved.

Another aspect of the invention resides in a method for the targeted alteration of a parent DNA strand (first strand, second strand) by contacting the parent DNA duplex with an oligonucleotide that contains at least one mismatch nucleotide compared to the parent strand, wherein the donor oligonucleotide contains a section that is methylated to a higher degree than the parent (acceptor) strand and/or wherein the parent strand is methylated to a lower degree of methylation, in the presence of proteins that are capable of targeted nucleotide exchange.

Thus, the inventive gist of the invention lies in the difference in the methylation status; of the intercalating oligonucleotide (sometimes referred to as the donor) and/or the modification (de-methylation) of one or both the strand(s) of the DNA duplex (sometimes referred to as the acceptor strand).

### DETAILED DESCRIPTION OF THE INVENTION

In one aspect, the invention pertains to an oligonucleotide for targeted alteration of a duplex DNA sequence. The duplex DNA sequence contains a first DNA sequence and a second DNA sequence. The second DNA sequence is the complement of the first DNA sequence and pairs to it to form a duplex. The oligonucleotide comprises a domain that comprises at least one mismatch with respect to the duplex DNA sequence to be altered. Preferably, the domain is the part of the oligonucleotide that is complementary to the first strand, including the at least one mismatch.

Preferably, the mismatch in the domain is with respect to the first DNA sequence. The oligonucleotide comprises a section that is methylated to a higher degree than the (corresponding part of the) second DNA sequence. In a certain embodiment, the second DNA sequence is methylated to a lower degree than the (corresponding part of the) section on the oligonucleotide. In certain embodiments, both the acceptor strand and the oligonucleotide are not methylated or are methylated to the same extent, such that no distinction can be made between the donor strand and the parental strand. This may remove any strand bias and render the targeted nucleotide exchange to a statistical process as any mechanism involving methylation can no longer distinguish between the two strands.

The domain that contains the mismatch and the section with a certain degree of methylation may be overlapping. Thus, in certain embodiments, the domain containing the mismatch is located at a different position on the oligonucleotide than the section of which the methylation degree is considered. In certain embodiments, the domain incorporates the section. In certain embodiments the section can incorporate the domain. In certain embodiments the domain and the section are located at the same position on the oligonucleotide and have the same length i.e. they coincide in length and position. In certain embodiments, there can be more than one section within a domain.

For the present invention, this means that the part of the oligonucleotide that contains the mismatch which is to be incorporated in the DNA duplex can be located at a different position from the part of the oligonucleotide where, in certain embodiments wherein the cell's repair system, or at least the proteins involved with this system, or at least proteins that are involved in TNE, determine which of the strands contain the mismatch and which strand is to be used as the template for the correction of the mismatch.

In certain embodiments, the oligonucleotide comprises a section that contains at least one, preferably at least 2, more preferably at least 3 methylated nucleotide(s) more than the corresponding part of the second strand and/or wherein the second strand contains at least one, preferably at least 2, more preferably at least 3 methylated nucleotide(s) less than the corresponding part of the section. In certain embodiments, the section on the oligonucleotide can contain more than 4, 5, 6, 7, 8, 9, or 10 methylated nucleotides. In certain embodiments the section is fully (C—) methylated. In certain embodiments the second strand is not methylated, at least at the position (or over the length of the section complementary to the first strand) of the oligonucleotide.

In certain embodiments, more than one mismatch can be introduced, either simultaneously or successively. The oligonucleotide can accommodate more than one mismatch on either adjacent or removed locations on the oligonucleotide. In certain embodiments the oligonucleotide can comprise two, three, four or more mismatch nucleotides which may be adjacent or remote (i.e. non-adjacent). The oligonucleotide can comprise further domains and sections to accommodate this, and in particular can comprise several sections. In certain embodiments, the oligonucleotide may incorporate a potential insert that is to be inserted in the acceptor strand. Such an insert may vary in length from more than five up to 100 nucleotides. In a similar way in certain embodiments, deletions can be introduced of similar length variations (from 1 to 1.00 nucleotides).

In certain embodiments, the part of the oligonucleotide that is complementary to the acceptor strand is methylated. The donor oligonucleotide may contain other sections than this part wherein the cytosines are not methylated.

In a further aspect of the invention, the design of the oligonucleotide can be achieved by:

(a) determining the sequence of the acceptor strand, or at least of a section of the sequence around the nucleotide to be exchanged. This can typically be in the order of at least 10, preferably 15, 20, 25 or 30 nucleotides adjacent to the mismatch, preferably on each side of the mismatch, (for example GGGGGGXGGGGG, wherein X is the mismatch);
(b) designing a donor oligonucleotide that is complementary to one or both the sections adjacent to the mismatch and contains the desired nucleotide to be exchanged (for example CCCCCYCOCCCC);
(c) providing (e.g. by synthesis) the donor oligonucleotide with methylation at desired positions. Methylation may vary widely, depending on the circumstances. Examples are C^{m}C^{m}C^{m}C^{m}C^{m}C^{m}YC^{m}C^{m}C^{m}C^{m}C^{m}C^{m}, C^{m}CC^{m}CC^{m}CYC^{m}CC^{m}CC^{m}C, CCCCCCYC^{m}C^{m}C^{m}C^{m}C^{m}C^{m}, C^{m}C^{m}C^{m}C^{m}C^{m}C^{m}YCCCCCC, CCCCCC^{m}YC^{m}CCCCC, C^{m}CCCCCYC^{m}CCCCC, C^{m}CCCCCYCCCCCC^{m}, C^{m}CCCCCYCCCCCC, and so on, wherein C^{m} stands for a methylated cytosine residue. For a different acceptor sequence, e.g. ATGCGTACXGTCCATGAT, corresponding donor oligonucleotides can be designed, e.g. TACGCATGYCAGGTACTA with methylation as variable as outlined hereinbefore, e.g. TACmGCmATGYCmAGGTACmTA (fully methylated), TACmGCATGYCmAGGTACmTA, TACGCmATGYCnAGGTACmTA TACmGCmATGYCAGGTACmTA, TACmGCATCYCmAGGTACTA (partly methylated);
(d) subjecting the DNA to be modified with the donor oligonucleotide in the presence of proteins that are capable of targeted nucleotide exchange, for instance, and in particular, proteins that are functional in the mismatch repair mechanism of the cell.

Without being bound by any theory, it is thought that the cells mismatch repair system may play a role in the method of the invention. However, the present invention is based on the use of methylated nucleotides in oligonucleotides in TNE, without further concern as to possible pathways. However, for a plausible theoretical background it is thought that, in certain embodiments, the cell's repair mechanisms relies on the co-operation of three proteins or their dimers. The first protein recognises the mismatch in the parent strand (e.g. MutS), the second is capable of nicking the mismatched strand (e.g. MutH). The third functions as a bridge between the first and second protein (e.g. MutL). In this embodiment, the relevant methylated section is located at or near the recognition site for an enzyme that is active in the cell's repair system. When the first protein recognises a mismatch, the third protein searches for the recognition site for the endonuclease. Once found, it is determined which of the two strands is the original parent strand and which is the strand that contains the mismatch on the basis of their methylation status. The strand containing the mismatch is nicked and digested in the direction of the mismatch with an exonuclease. After digestion of the mismatch position, the cell's repair mechanism will then fill in the missing nucleotides based on the determined original parent strand, thereby incorporating the correct nucleotide at the position of the mismatch. By using intercalating oligonucleotides that are more densely methylated at the relevant position or by demethylating the original strand, the repair mechanism can be used to incorporate the desired nucleotide at the position of the mismatch and thereby effectively providing for targeted nucleotide exchange (TNE). See also FIG. 2.

The delivery of the oligonucleotide can be achieved via electroporation or other conventional techniques that are capable of delivering either to the nucleus or the cytoplasm. In vitro testing of the method of the present invention can be achieved using the Cell Free system as is described i.e. in WO01/87914, WO03/027265, WO99/58702, WO01/92512.

As used herein, the degree of methylation, also denoted with MDF, is a parameter that can be used to indicate, for a given oligonucleotide or (part of) a DNA strand, the relative number of positions that are methylated, i.e. contain a nucleotide that is methylated. The methylation degree factor or MDF is herein defined as:

$$MDF = \frac{\begin{matrix}Number of possible positions \\ that are methylated\end{matrix}}{\begin{matrix}Number of positions \\ that can be methylated\end{matrix}}$$

Thus, when all available positions on the DNA sequence or oligonucleotide of interest are methylated, MDF equals 1. If no methylation is present in the strand, MDF equals 0. MDF generally will be between 0 and 1. Note that the definition of MDF is in principle independent of the length of the nucleotide strand that is compared. However, when MDFs of different strands are compared it is preferred that the strands have about the same length or that sections of comparable length are taken Note that MDF does not take into account that methylation can be grouped together on a strand. A higher degree of methylation of ascertain strand A compared to a strand B thus means that MDF(A)>MDF(B). For upstream and downstream sections, corresponding MDF values may be used. To accommodate the effect of the position of the methylated nucleotide a weighing actor can be introduced into the MDF value. For instance, the effect of a methylated nucleotide on the donor oligonucleotide adjacent to the mismatch can be larger than that of a methylated nucleotide that is located at a distance five nucleotides removed from the mismatch. In the context of the present invention, MDF (Donor)>MDF (Acceptor) or MDF (Donor)/MDF (Acceptor)>1.0.

In certain embodiments, the oligonucleotide is methylated wherein the ratio of methylation of the oligonucleotide to the corresponding section on the duplex MDF (Donor)/MDF (Acceptor)>1.0, preferably >1.002, more preferably >1.02, even more preferably >1.05. Particularly preferred is >1.1, more preferred is >1.2. Particular preference is given to MDF (Donor)/MDF (Acceptor)>1.2, preferably >1.3, more preferably >1.5, most preferably >2.0, but ratio's such as >5 or 10 are more preferred. The degree of methylation of the donor and/or the acceptor can be expressed in terms relative to full methylation, i.e. full methylation is set at 100% methylation of the cytosines in the area wherein the oligonucleotide is complementary to the first strand (the domain). This means that in certain embodiments the degree of methylation can be quantified as 99, 98, 97, 96, 95, 90, 85, 80, 75, 70, 60, 50, 40, 30, 20 or even 10% compared to a fully methylated strand. In the earlier described embodiment wherein both strands and the oligonucleotide are not methylated, the degree of methylation can be as low as 0%.

It is further observed that certain organisms (such as certain strains of E. Coli and Salmonella bacteria) also express adenine methylation. The present invention is likewise applicable to such organisms.

In certain embodiments of the invention, the nucleotide in the oligonucleotide at the position of the mismatch can be methylated. Whether or not the mismatch can be methylated will depend to a large extent on the exact mechanism of the targeted nucleotide exchange or of the cell's DNA repair mechanism using the difference in methylation between the donor and acceptor strands. The same holds for the exact location of the other methylated positions in the neighborhood or vicinity of the mismatch. However, based on the disclosure presented herein, such an oligonucleotide can be readily designed and tested, taking into account the test procedures for suitable oligonucleotides as described herein elsewhere (Cell Free System, which is available for yeast, plant and human applications alike). In certain embodiments, the nucleotide at the position of the mismatch is not methylated. In certain embodiments, full or partial methylation is adjacent to the mismatch, preferably within 2, 3, 4, 5, 6 or 7 nucleotides of the mismatch. In certain embodiments, methylation is located at a position downstream from the mismatch, for instance at a position 10, 15, 20, 25, 30, 35 or even more than 40 nucleotides removed from the mismatch. In certain embodiments, methylation is located at a position upstream from the mismatch for instance at a position 10, 15, 20, 25, 30, 35 or even more than 40 nucleotides removed from the mismatch. In certain embodiments, the methylation is located up and/or downstream from 10 bp to 500 bp from the mismatch, preferably from 50 to 200 bp, more preferably from 100 to 150 from the mismatch.

The oligonucleotides that are used as donors can vary in length, but generally vary in length between 10 and 500 nucleotides, with a preference for 11 to 100 nucleotides, preferably from 15 to 90, more preferably from 20 to 70 most preferably from 30 to 60 nucleotides. In certain embodiments the oligonucleotide are designed such that they are part DNA and part RNA, preferably 5′-RNA-DNA.

In one aspect, the invention pertains to a method for the targeted alteration of a duplex acceptor DNA sequence, comprising combining the duplex acceptor DNA sequence with a donor oligonucleotide, wherein the duplex acceptor DNA sequence contains a first DNA sequence and a second DNA sequence which is the complement of the first DNA sequence and wherein the donor oligonucleotide comprises a domain that comprises at least one mismatch with respect to the duplex acceptor DNA sequence to be altered, preferably with respect to the first DNA sequence, and wherein a section of the donor oligonucleotide is methylated to a higher degree than the second DNA sequence and/or wherein the second DNA is methylated to a lower degree of methylation than the corresponding section of the donor oligonucleotide, in the presence of proteins that are capable of targeted nucleotide exchange.

The invention is, in its broadest form, generically applicable to all sorts of cells such as human cells, animal cells, plant cells, fish cells, reptile cells, insect cells, fungal cells, bacterial cells and so on. The common denominator appears that the organism has a mismatch repair mechanism that is sensitive to the difference in methylation between the two strands of DNA.

The invention is applicable for the modification of any type of DNA, such as DNA derived from genomic DNA, linear DNA, artificial chromosomes, nuclear chromosomal DNA, organelle chromosomal DNA, BACs, YACs. The invention can be performed in vivo as well as ex vivo.

The invention is, in its broadest form, applicable for many purposes for altering a cell, correcting a mutation by restoration to wild type, inducing a mutation, inactivating an enzyme by disruption of coding region, modifying bioactivity of an enzyme by altering coding region, modifying a protein by disrupting the coding region.

The invention also relates to the use of oligonucleotides essentially as described hereinbefore, for altering a cell, correcting a mutation by restoration to wild type, inducing a mutation, inactivating an enzyme by disruption of coding region, modifying bioactivity of an enzyme by altering coding region, modifying a protein by disrupting the coding region, mismatch repair, targeted alteration of (plant) genetic material, including gene mutation, targeted gene repair and gene knockout

The invention further relates to kits, comprising one or more oligonucleotides as defined herein elsewhere, optionally in combination with proteins that are capable of inducing MMR, and in particular that are capable of TNE.

The invention further relates to modified genetic material obtained by the method of the present invention, to cells and organisms that comprise the modified genetic material, to plants or plant parts that are so obtained.

### DESCRIPTION OF THE FIGURES

FIG. 1: Schematic representation of targeted nucleotide exchange. An acceptor duplex DNA strand containing a nucleotide that is to be exchanged (X) is brought into contact with a donor oligonucleotide (schematically given as NNN^{m}NNN^{m}YNN^{m}NN^{m}) containing the nucleotide to be inserted (Y). The triplex structure is subjected to or brought into contact with an environment that is capable of TNE or at least with proteins that are capable of performing TNE, such as are known as the cell-free enzyme mixture or a cell-free extract (see i.a. WO99/58702, WO01/73002).

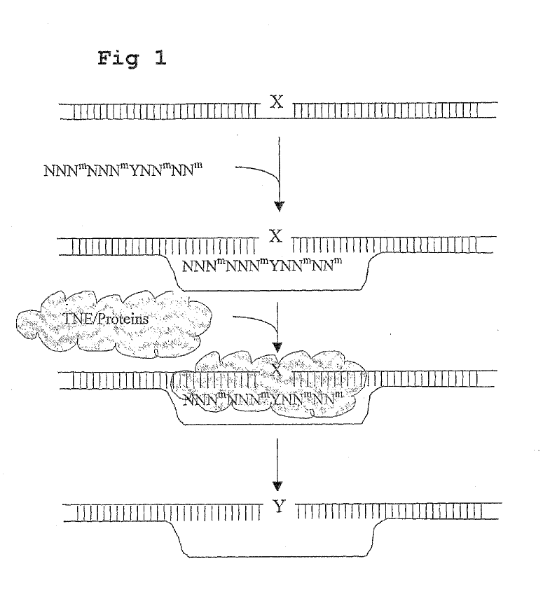

FIG. 2: schematic representation of theoretical models of mismatch repair for:

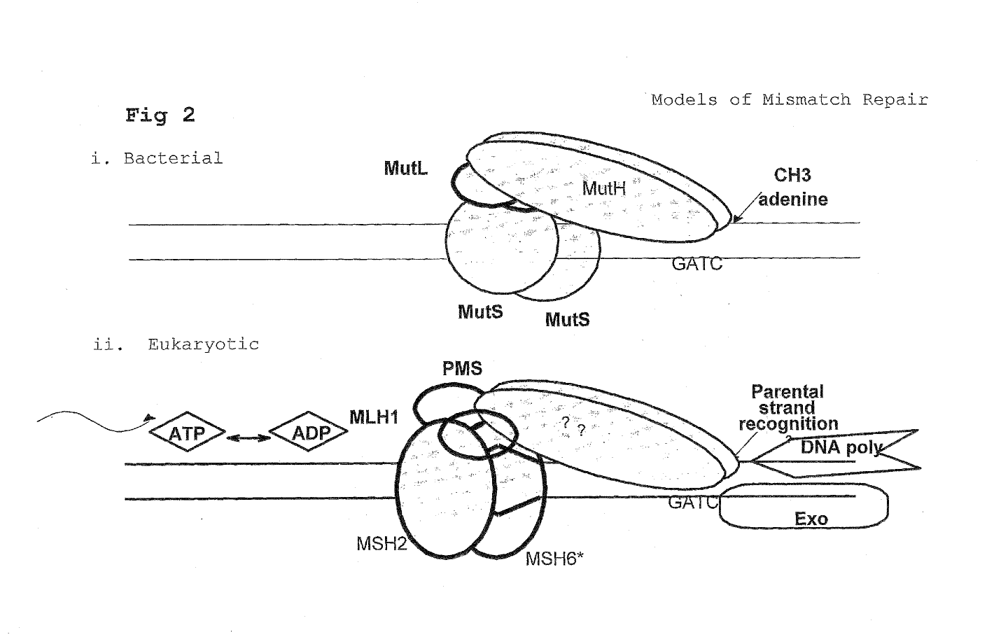

I). Bacteria

The MutS dimer binds to base-base mismatches and to 1-4 looped out nucleotides in the DNA duplex. MutS recruits two other proteins, MutL and MutH dimers. MutH nicks the DNA strand at the hemi-methylated GATC site. The strand that carries a methylated adenine is regarded as the parental strand. MutL dimers form a bridge between MutS and MutH.

II). Eukaryotes

MutSa dimer (MutSa=MSH2/MSH6*) binds base-base-mismatches, short loops +1 Indel. MutSb diner (MutSb=MSH2/MSH3*) binds +1-12 Indels. Mismatch is co-factor for release of ADP in exchange for ATP. ATP Bound state of MSHa binds firmly to mismatch. DNA poly d/e 3′-5′ exonuclease degrades misincorporation on the leading strand. EXo1 5′-3′ exonuclease degrades misincorporation on the lagging strand.

### EXAMPLES

### Example 1

### TNE of the PPO Gene in Arabidopsis

### Description of ddm Mutant Arabidopsis

DNA methylation requires the action of additional factors. The first to be identified was the DDM-1 (decrease in DNA methylation) (Vongs et al. 1993 Science 26:1926-1928) Homozygous ddm1-2 plants showed a 70% decrease in DNA methylation. Hypomethylation was observed initially in repeated sequences, and then after several generations of selfing in single copy DNA (Vongs et al. 1993 Science 26:1926-1928; Kakutani et al. 1996 Proc. Natl. Acad. Sci. USA 93: 12406-12411). The DDM-1 gene encodes a member of the SNF2/SWI2 family DNA dependent ATPases (Jeddeloh et al. 1999 Nat. Genet, 22: 94-97). DDM-1 appears to be involved in chromatin remodelling, allowing the methyltransferase to access DNA.

Protoplast Isolation from the Arabidopsis DDM-1 Mutant

For the isolation of protoplasts from Arabidopsis DDM-1 mutants, seeds from the DDM-1 mutant are sterilized for 15 minutes in a sterilization solution (10% hypochlorite solution, 0.1% Triton X-100) and washed repeatedly with sterile water. Seeds are then placed on 0.5×BM agar plates (MS medium containing BS vitamins, 0.8% agar and 3% sucrose) for germination. Fifteen to twenty 7 day old seedlings are transferred into 50 ml of liquid BM medium and shaken at 120 rpm at 25° C. using 16 hr light and 8 hr dark period. After 10˜14 days growth, the plantlets are removed and the roots are separated from the green tissue. They are cut into small root segments (2-4 mm) and transferred to MSAR-1 medium (BM medium containing 2.0 mg/L indole-3-acetic acid (IAA), 0.5 mg/L 2,4-dichloro-phenoxyacid (2,4-D), 0.5 mg/L 6-(γ,γ-dimethylallylamino) purine riboside (IPAR). The Petri dishes are placed on a shaker set at 100 rpm in the dark for 7-12 days. The liquid medium is removed from the Petri plates and the explants washed with 0.45M sucrose solution. The sucrose is removed and 20 ml of enzyme solution is added (1% cellulose (Onozuka R-10; Serva, Heidelberg, Germany), 0.25% macerozyme (R10; Serva) in PM medium (0.5×BM medium containing MSAR-1 hormones with 0.45M sucrose or 0.45M mannitol). The root explants are incubated for 12-16 hrs with occasional shaking. The protoplasts are collected with a pipette and sieved through 100-, 50-, and 25 μm sieves. The protoplast suspension is centrifuged for 5 min at 50×g. The band of floating protoplasts concentrated at the top of the solution is collected and transferred into a new tube. The protoplasts are resuspended in 0.45M mannitol solution and the cells pelleted at 60×g for 5 minutes. The protoplasm are washed by repeating this step twice. The protoplasts are resuspended in electroporation solution.

### PPO Target Gene and Design of Methylated Chimeric or Single Stranded Oligonucleotides

Arabidopsis protoplasts are transfected with a chimeric or single stranded oligonucleotide designed to introduce a single nucleotide change in the Arabidopsis PPO gene. The PPO protein residing in the chloroplast is the target of the herbicide butafenacil that competitively blocks the activity of the enzyme. This results in accumulation and leakage of protoporphyrinogen IX into the cytoplasm where it results in rapid cellular damage and plant cell death. The mutations S305L and Y426M render Arabidopsis resistant to butafenacil (Hanin et al. 2001. Plant J. 28:1-8). Chimeric oligonucleotides designed to produce these mutations are thus introduced into Arabidopsis DDE-1 protoplasts and butafenacil resistant plants can be selected during plant regeneration.

For production of the S305L mutation, TCA (serine) to TTA (leucine), the chimeric (AtCPPOS305L) and single stranded (AtPPOS305L) oligonucleotides have the following sequence.

### AtCPPOS305L

[SEQ ID NO: 1]

5′TTCCAAGCTCTTAGCTATCACTAATTTTuuagugauagCTAAGagcuu
ggaaGCGCGTTTCGCGC 3′
AtPPOS305L

[SEQ ID NO: 2]

5′ATGTTAAGTTGTATCCTCCGCTCTCCAGCTTAGTGATACCTAAGAGCT
TCCAAGACAACTTAACTTTGCTACCTAATCTT 3′

(uppercase: DNA, C: 5-methyl cytosine, lower case: 2′-O-methyl RNA residues. The mismatch nucleotides are underlined)

For production of the Y426M mutation (TAC to ATG), the chimeric (AtCPPOY426M) and single stranded (AtPPOY426M) oligonucleotides have the following sequence:

AtCPPOY426M

[SEQ ID NO: 3]

5′CGCTGTTGAACATGATTGGCGGGTCTTTTgacccgccaaTCATGuuca
acagcaGCGCGTTTTCGCGC 3′
AtPPOY426M

[SEQ ID NO: 4]

5′GACTTGGACAGAATTCCGGTGTTTGTAGACCCGCCAATCATGTTCAAC
AGCAAAATTCTTCCGGGCGGTGC 3′

(uppercase: DNA, C: 5-methyl cytosine, lower case: 2′-O-methyl RNA residues. The mismatch nucleotides are underlined)

### Protoplast Electroporation

Using PHBS as an electroporation medium (10 mM Hepes, pH 7.2; 0.2 M mannitol, 150 mM NaCL; 5 mM CaCL2) and with a density of protoplasts in the medium during electroporation of ca. 1×10⁶/ml, the electroporation settings are 250V (625 V cm⁻¹) charge and 800 μF capacitance with a recovery time between pulse and cultivation of 10 minutes. For each electroporation ca. 1-2 μg oligonucleotide is used and 20 μgr plasmid per 800 microliter electroporation=25 μg/ml.

### Protoplast Regeneration and Selection in Butafenacil

After electroporation the cells are pelleted again at 60×g for 5 minutes and resuspended in 1 ml alginate solution (1% (w/v) sodium alginate solution in BM medium containing 0.45M sucrose) at a density of 3-5×10⁵ cells/ml and 200-500 μl drops are created on calcium agar plates (20 mM calcium chloride, 0.45M sucrose and 1% agarose). The drops of alginate gel-carrying protoplasts are transferred into 55 mm petri dishes containing 5 ml PM medium and the protoplasts are cultured in a growth chamber at 25° C. under dim light. After 7 and 14 days 2.5 ml of the spent medium is removed and fresh PM medium added. 1 ml of PM medium is removed and 1 ml MSAR I medium added on days 21, 28, and 35, at which point microcalli have formed. At this point, herbicide resistant calli are selected by addition of 50 nM butafenacil to the culture medium.

### PCR Amplification of the PPO Gene

Butafenacil resistant callus is analyzed as follows. DNA is isolated from growing callus using the DNeasy Plant DNA Kit following the manufacturer's instructions. PCR is then done using primers (5′-GCATAATAGGTGGTACTTTT [SEQ ID No:5] and 5′-GCTGCAACTGGTGGGTAATA [SEQ ID No:6]) to amplify a 370 bps fragment of the PPO gene including codon 305. As the chimeric oligonucleotide is expected to convert only one copy of the PPO gene, these primers amplify both the wild type and converted PPO locus. Similarly, PCR is performed using the following primers (5′ TAATGACGGTGCCATCTCAT [SEQ ID NO:7] & 5′ CTAGAAACTGAGGAATGGCT [SEQ ID NO:8]) that amplify a 436 bps region of the Arabidopsis PPO sequence including codon 426.

### Sequencing to Prove the Nucleotide Conversion

The amplified PCR fragments are sequenced. Callus that had undergone a successful nucleotide conversion as expected show a double peak at the second position of codon 305 (TCA and TTA) and at all positions in codon 426 (TAC to ATG).

### Example 2

### TNE of the ALS Gene in Arabidopsis

Arabidopsis protoplasts are produced as described in example 1. The chimera AtALSCW574L or single stranded oligonucleotide AtALSW574L are introduced into the protoplasts to promote the change of codon W574 (TGG) to leucine (TTG) in the gene coding for acetolactate synthase (ALS). Similarly, conversion of codon 197 (proline, CCT) to glutamic acid (CAG) can be achieved by using the chimera AtALSCP197Q or the single stranded oligonucleotide AtALSP197Q. These amino acid substitutions have been shown to confer resistance to the herbicide chlorsulfuron.

AtALSCW574L

[SEQ ID NO: 9]

5′GTTATGCAATTGGAAGATCGGTTTTTTaaccgaucuuCCAATugcaua
acGCGCGTTTTCGCGC 3′
AtALSW574L

[SEQ ID NO: 10]

5′AATGTGTGAGCTCGGTTAGCTTTGTAGAACCGATCTTCCAATTGCATA
ACCATGCCAAGATGCTGGTTGTTAATAAAAG 3′
AtCALSP197Q

[SEQ ID NO: 11]

5′CAGGACAAGTCCAGCCGTCGTATGATTTTucauacgacgGCTGGacuu
guccugGCGCGTTTTCGCGC 3′
AtALSP197Q

[SEQ ID NO: 12]

5′CGGAGTCTCTTGAAACGCATCTGTACCAATCATACGACGCTGGACTTG
TCCTGTGATTGCTACAAGAGGAACACTATCT 3′

(uppercase: DNA, C: 5-methyl cytosine, lower case: 2′-O-methyl RNA residues. The mismatch nucleotides are underlined.)

### Example 3

### TNE of the PPO Gene in Arabidopsis Using Methylated Oligonucleotides

The experiment is performed as in example 1, except the oligonucleotide is a single stranded oligonucleotide with phosphorothioate residues.

The methylated single stranded oligonucleotide for conversion of the Arabidopsis PPO S305 codon to leucine has the following sequence.

[SEQ ID NO: 13]

5′AAGTTGTATCCTCCGCTCTCCAGCTTAGTGATACCTAAGAGCTTCCAA
GACAACTTAACTTTGCTACCTAATCTTGCAGA 3′.

The ‘c’ nucleotides are 5-methylcytosine and the remaining nucleotides are standard DNA. The mismatch nucleotide is underlined. In order to improve the nuclease resistance of the oligonucleotide phosphorothioate linkages can be included at the ends of the oligonucleotide. The nucleotides linked in this manner are indicated in bold type.

### Example 4

### TNE in Tobacco

### Tobacco Shoot Cultures

The source material for this example is tobacco SR1 in vitro shoot cultures. They are grown under sterile conditions in large glass jars (750 ml capacity) in MS20-medium in growth chambers at a temperature of 25/20° C. (day/night) and a photon flux density of 80 μE.m⁻².s⁻¹ with a photoperiod of 16/24 h. MS20 medium is basic Murashige and Stooges medium (Murashige, T. and Skoogr, F., Physiologia Plantarum, 15: 473-497, 1962) containing 2% (w/v) sucrose, no added hormones and 0.8% Difco agar. The shoots are subcultured every 3 weeks to fresh medium.

### Demethylation Via Azacytidine

Two weeks prior to protoplast isolation, the tobacco in vitro shoot cultures are subcultured on MS20 medium containing 75 μg/ml 5-azacytidine in order to demethylate the target locus DNA.

### Protoplast Isolation

For the isolation of mesophyll protoplasts, fully expanded leaves of 3-6 week old shoot culture plants are harvested. The leaves are carefully sliced, through the lower epidermis and from the midrib outward, into 1 mm thin strips. The sliced leaves are transferred to large (100 mm×100 mm) Petri dishes containing 45 ml MDE basal medium for a preplasmolysis treatment of 30 min. MDE basal medium contained 0.25 g KCl, 1.0 g MgSO₄.7H₂O, 0.136 g of KH₂PO₄, 2.5 g polyvinylpyrrolidone (MW 10,000), 6 mg naphthalene acetic acid and 2 mg 6-benzylaminopurine in a total volume of 900 ml. The osmolality of the solution is adjusted to 600 mOsm.kg⁻¹ with sorbitol, the pH to 5.7.

After preplasmolysis, 5 ml of enzyme stock is added to each Petri dish. The enzyme stock consists of 750 mg Cellulase Onozuka R10, 500 mg driselase and 250 mg macerozyme R10 per 100 ml, filtered over Whatman paper and filter-sterilized. The Petri dishes are sealed and incubated overnight in the dark at 25° C. without movement to digest the cell walls.

Next morning, the dishes are gently swirled to release the protoplasts. The protoplast suspension is passed through 500 μm and 100 μm sieves into 250 ml Erlenmeyer flasks, mixed with an equal volume of KCl wash medium, and centrifuged in 50 ml tubes at 85×g for 10 min. KCl wash medium consisted of 2.0 g CaCl₂.2H₂O per liter and a quantity of KCl to bring the osmolality to 540 mOsm.kg⁻¹.

The protoplasts, recovered from the centrifugation pellets, are resuspended in MLm wash medium and centrifuged once again in 10 ml glass tubes at 85×g for 10 min. MLm wash medium contained the macro-nutrients of MS medium (ref) at half the normal concentration, 2.2 g of CaCl₂.2H₂O per liter and a quantity of mannitol to bring the osmolality to 540 mOsm.kg⁻¹.

The protoplasts, recovered from the pellets of this second centrifugation step, are resuspended in MLs wash medium and centrifuged again in 10 ml glass tubes at 85-100×g for 10 min. MLs wash medium contained the macro-nutrients of MS medium (ref) at half the normal concentration, 2.2 g of CaCl₂.2H₂O per liter and a quantity of sucrose to bring the osmolality to 540 mOsm.kg⁻¹.

The protoplasts are recovered from the floating band and resuspended in an equal volume of KCl wash medium. Their densities are counted using a haemocytometer. Subsequently, the protoplasts are centrifuged again in 10 ml glass tubes at 85×g for 5 min and the pellets resuspended at a density of 1×10⁵, protoplasts ml⁻¹ in electroporation medium. All solutions are kept sterile, and all manipulations are done under sterile conditions.

### PPO and ALS Target Genes and Design of Methylated Chimeric and Single Stranded Oligonucleotides

The following single stranded oligonucleotide NtS316L is designed to introduce a serine to leucine conversion (TCT to TTA) at S316 of the tobacco protoporphyrinogen oxidase gene (Gene Bank Accession NTPPOY13465). Similarly, the following single stranded oligonucleotide NtPPOY437M can be used to introduce a tyrosine to methionine conversion at Y437 in the tobacco protoporphyrinogen oxidase protein. In the tobacco acetolactate synthase (ALS) SurA gene (Gene Bank Accession X07644) the amino acid conversions P194Q and W571L make the ALS protein insensitive to the sulfonylurea herbicide chlorsulfuron. The single stranded oligonucleotides NtALSP1940 and NtALSW571L can be used to introduce these single nucleotide changes at the tobacco ALS gene by TNE. The chimeras NtCALSP194Q and NtCALSW571L can also be used to introduce these mutations.

NtPPOS316L

[SEQ ID NO: 14]

5′GTATGTCAAGTGATATCCTCCTTTTTCTGACTTAGTAATGCTTAAAAG
CTTCCATGATAGTTTTAATTTGCTTCCCAATC 3′
NtPPO437M

[SEQ ID NO: 15]

5′CGTCTTAGACAAAATTTCAGGATTTTTTGCTCCTCCAATCATGTTCAA
GAGTAGCACCCGACCTTTTGGGGCACGGTTAG 3′
NtALSP194Q

[SEQ ID ND: 16]

5′CAACAATAGGAGTTTCCTGAAAAGCATCAGTACCTATCATCCTACGTT
GCACTTGACCTGTTATAGCAACAATGGGGAC 3′
NtALSW571L

[SEQ ID NO: 17]

5′CCCCAGGTATGTGTGTGCTCTGTTAGCCTTATAGAACCGATCCTCCAA
TTGAACCACCATTCCCAAGTGTTGATTATTCA 3′

The cytosine nucleotides in bold are 5-methylcytosine while the remaining nucleotides are normal DNA. The mismatch nucleotides are underlined.

The nucleotide conversions can also be introduced using chimeras. The chimera NtCPPOS316L can introduce the tobacco protoporphyrinogen S3161conversion, while the chimera NtCPPOY437M can similarly perform the Y437M conversion. Similarly, the chimeras NtCALSP194Q and NtCALSW571L can be used to introduce the P194Q and W571L mutations that lead to tobacco lines resistant to the herbicide chlorsulfuron.

NtCPPOS316L

[SEQ ID NO: 13]

5′ATGGAAGCTTTTAAGCATTACTAAGTTTTcuuaguaaugCTTAAaagc
uuccauGCGCGTTTTCGCGC 3′
NtCPPOY437M

[SEQ ID NO: 19]

5′ACTCTTGAACATGATTGGAGGAGCTTTTgcuccuccaaTCATGuucaa
gaguGCGCGTTTTCGCGC 3′
NtCALSP194Q

[SEQ ID NO: 20]

5′CAGGTCAAGTGCAACGTAGGATGATTTTTaucauccuacGTTGCacuu
gaccugGCGCGTTTTCGCGC 3′
NtCALSW571L

[SEQ ID NO: 21]

5′TGGTGGTTCAATTGGAGGATCGGTTTTTTaaccgauccuCCAATugaa
ccaccaGCGCGTTTTCGCGC 3′

(uppercase: DNA, C: 5-methylcytosine, lower case: 2′-O-methyl RNA residues. The mismatch nucleotides are underlined.)

### Protoplast Electroporation

Using PHBS as an electroporation medium (10 mM Hepes, pH 7.2; 0.2 M mannitol, 150 mM NaCL; 5 mM CaCL2) and with a density of protoplasts in the medium during electroporation of ca. 1×10⁶/ml, the electroporation settings are 250V (625 V cm⁻¹) charge and 800 μF capacitance with a recovery time between pulse and cultivation of 10 minutes. For each electroporation ca. 1-2 μg oligonucleotide are used and 20 μg plasmid per 800 microliter electroporation=25 μg/ml.

### Protoplast Regeneration and Selection in Butafenacil or Chlorsulfuron

After the electroporation treatment, the protoplasts are placed on ice for 30 min to recover. They are then resuspended in T₀ culture medium at a density of 1×10⁵ protoplasts ml⁻¹. To culture medium contained (per liter, pH 5.7) 950 mg KNO₃, 825 mg NH₄NO₃, 220 mg CaCl₂.2H₂O, 185 mg MgSO₄.7H₂O, 85 mg KH₂FO₄, 27.85 mg FeSO₄.7H₂O, 37.25 mg Na₂EDTA.2H₂O, the micro-nutrients according to Heller's medium (Heller, R., Ann Sci Nat Bot Biol Veg 14: 1-223, 1953), vitamins according to Morel and Wetmore's medium (Morel, G. and R. H. Wetmore, Amer. J. Bot. 38: 138-40, 1951), 2% (w/v) sucrose, 3 mg naphthalene acetic acid, 1 mg 6-benzylaminopurine and a quantity of mannitol to bring the osmolality to 540 mOsm.kg⁻¹.

The protoplasts resuspended in T₀ culture medium are then mixed with an equal volume of a solution of 1.6% SeaPlaque Low Melting Temperature Agarose in T₀ culture medium, kept liquid after autoclaving in a waterbath at 30° C. After mixing, the suspension is gently pipetted in 2.5 ml aliquots into 5 cm Petri dishes. The dishes are sealed and incubated at 25/20° C. (16/24 h photoperiod) in the dark.

After 8-10 days incubation in the dark, the agarose medium is cut into 6 equal pie-shaped parts, which are transferred to 10 cm Petri dishes each containing 22.5 ml of liquid MAP₁AO medium. This medium consisted of (per liter, pH 5.7) 950 mg KNO₃, 825 mg NH₄NO₃, 220 mg CaCl₂.2H₂O, 185 mg MgSO₄.7H₂O, 85 mg KH₂PO₄, 27.85 mg FESO₄.7H₂O, 37.25 mg Na₂EDTA.2H₂O, the micro-nutrients according to Murashige and Skoog's medium (Murashige, T. and Skoog, F., Physiologia Plantarum, 15: 473-497, 1962) at one tenth of the original concentration, vitamins according to Morel and Wetmore's medium (Morel, G. and R. H. Wetmore, Amer. J. Bot. 38: 138-40, 1951), 6 mg pyruvate, 12 mg each of malic acid, fumaric acid and citric acid, 3% (w/v) sucrose, 6% (w/v) mannitol, 0.03 mg naphthalene acetic acid and 0.1 mg 6-benzylaminopurine. For purposes of selection of colonies with a successful base conversion, 140 nM butafenacil or 41 nM chlorsulfuron is also added to the medium. The Petri dishes are incubated at 25/20° C. in low light (photon flux density of 20 μE.m⁻².s⁻¹) at a photoperiod of 16/24 h. After two weeks, the Petri dishes are transferred to full light (80 μE.m⁻².s⁻¹). During this period of selection, most protoplasts died. Only protoplasts, in which through the action of the chimeric oligonucleotides a base change has occurred in the target gene so as to confer resistance to the herbicide, divide and proliferate into protoplast-derived microcolonies.

Six to eight weeks after isolation, the protoplast-derived colonies are transferred to MAP₁ medium. The agarose beads by this time fall apart sufficiently to transfer the microcolonies with a wide-mouthed sterile pipette, or else they are individually transferred with forceps. MAP₁ medium has the same composition as MAP₁AO medium, with however 3% (w/v) mannitol instead of 6%, and 46.2 mg.l⁻¹ histidine (pH 5.7). It was solidified with 0.8% (w/v) Difco agar.

After 2-3 weeks of growth on this solid medium, the colonies are transferred to regeneration medium RP, 50 colonies per 10 cm Petri dish. RP medium consisted of (per liter, pH 5.7) 273 mg KNO₃, 416 mg Ca(NO₃)₂.4H₂O, 392 mg Mg(NO₃)₂.6HO, 57 mg MgSO₄.7H₂O, 233 mg (NH₄)₂SO₄, 271 mg KH₂PO₄, 27.85 mg FeSO₄.7H₂O, 37.25 mg Na₂EDTA.2H₂O, the micro-nutrients according to Murashige and Skoog's medium (Murashige, T. and Skoog, F., Physiologia Plantarum, 15: 473-497, 1962) at one fifth of the published concentration, vitamins according to Morel and Wetmore's medium (Morel, G. and P. H. Wetmore, Amer. J. Bot. 38: 138-40, 1951), 0.05% (w/v) sucrose, 1.8% (w/v) mannitol, 0.25 mg zeatin and 140 nM butafenacil or 41 nM chlorsulfuror, and is solidified with 0.8% (w/v) Difco agar.

### PCR Amplification of Target Gene

DNA is isolated from butafenacil and chlorsulfuron resistant tobacco microcolonies using the DNeasy kit (Qiagen). Total tobacco DNA is then used as a template in the PCR reaction. For detection of the tobacco PPO S316L conversion the primers CATGAGGAATCAGTTGAGCA [SEQ ID NO: 22] and TTTGAAAGTGCATCTGCTGC [SEQ ID NO: 23] are used that amplify a 509 bps region of the tobacco PPO gene, including codon S316. Similarly, for detection of the tobacco PPO Y437M conversion the primers ACTAAGTCAGAAAAAGGAGGATATC [SEQ ID NO: 24] and AAGAGGATCTTCGAGCTTTGG [SEQ ID NO: 25] are used that amplify a 471 bps region of the tobacco PPO gene. Conversion of the targeted codons in the tobacco ALS gene are detected using the primers 5′GGTCAAGTGCCACGTAGGAT [SEQ ID NO:26] & 5′GGGTGCTTCACTTTCTGCTC [SEQ ID NO:27] that amplify a 776 bp fragment of this gene, including codon 194. The primers 5′CCCGTGGCAAGTACTTTCAT [SEQ ID NO:28] & 5′GGATTCCCCAGGTATGTGTG [SEQ ID NO:29] are likewise used to amplify 794 bps fragment of the tobacco ALS gene, including the codon 571.

### Sequencing to Proof Nucleotide Conversion

Nucleotide conversion in the herbicide resistant tobacco callus is confirmed by sequencing the PCR products obtained from such callus. Upon conversion of the tobacco PPO S316 codon (TCT to TTA) a double peak at the second and third nucleotides can be observed. Upon conversion of the tobacco PPO Y437 codon (TAC to ATG) double peaks at all nucleotide positions in this codon are observed. Similarly, conversion of the tobacco ALS P194 codon (CCA to CAA) results in a double peak at the second position of the codon (C/A). Finally, conversion of the tobacco ALS W571 codon (IGG to TTG) results in a double peak at the second codon position (G/T).

### Example 5

### TNE in Mammalian Cells

### Mouse Embryonic Stem Cell Culture

Mouse ES cells (Chemicon International) are maintained in HEPES-buffered (20 mM, pH 7.3) DMEM (Dulbecco's Modified Eagle Media) solution supplemented with 15% fetal calf serum (Amaxa Biosystems) 0.1 mM non-essential amino acids (Invitrogen), 0.1 mM b-mercaptoethanol (Sigma), and penicillin-streptomycin (Irvine Scientific). ES cells are grown on feeder layers of gamma-irradiated mouse embryonic fibroblast cells and supplemented with leukemia inhibitory factor (LIP; Amaxa Biosystems) at 106 U/ml to prevent ES cell differentiation. To remove feeder cells, cells are washed in PBS and detached from the plate by trypsinization (0.05% trypsin in PBS), quenched in trypsin with 5-fold media addition and briefly spun down. Cells are then plated onto a single 10 cm feeder-free dish and allowed to sit for 30 min. Non-adherent cells are then collected. 3×10⁶ cells are spun for 5 min at 80×g at 4° C. to remove the culture medium and resuspended in PBS.

Transfection of Cells

3×10⁶ Cells are electroporated in a 90 μl mixture of 20 mM HEPES (pH 7.0), 137 mM NaCl, 5 mM KCl, 0.7 mM Na₂HPO₄, 6 mM glucose, and 0.1 mM 8-mercaptoethanol, with a plasmid DNA and oligonucleotide mixture containing 2 μg oligos and 10 μg DNA (see below) at a set voltage of 400 V and a capacitance of 75 μF, in a 0.4 cm-diameter cuvette with a Bio-Rad GenePulser II. After transformation, cells plated on gelatin-coated dishes and cultured for 24-48 h in a humidified incubator at 37° C. Antibiotic selection is initiated on the following day using G418 (Invitrogen) at an active concentration of 350 μg/ml, and then increased to 500 μg/ml and continued for 8-15 days before picking transformant cells.

Plasmid and Oligonucleotide DNA

Plasmid DNA and oligos are purified prior to transfection with QIAGEN® EndoFree® Plasmid Kits [Cat. No. Giga Kit, 12362 Maxi Kit, 12381 Mega Kit]. The purified DNA is resuspended in TE buffer (10 mM Tris/HCl, 1 mM EDTA, pH 8.0) to a concentration between 1-5 μg/μl.

Controls: Transfection efficiency is determined with a plasmid expressing green fluorescent protein (pmaxGFP; Amaxa Biosystems). This is used at a concentration of 10 μg per transfection reaction.

### Plasmids and Oligonucleotides

Plasmid pCMVNeoFlAsH from Aurora BioSciences is used in these experiments. It contains a neomycin cassette driven by the SV40 early promoter and can be maintained episomally in mammalian cells. We introduce a point mutation at codon 31 of the neomycin gene (TGC to TGA) thus introducing a stop codon at this position and inactivating the neomycin gene. This is then transfected to mouse ES cells and clones containing this construct are selected using zeocin (100 μg/ml). It is confirmed that these clones are also G418 sensitive. One clone is selected for transfection with the single stranded oligonucleotide Neo(+) or the chimeric oligonucleotide CNeo(+), both designed to convert the stop codon back to its original codon sequence. The sequence of these is shown below.

Neo(+)

[SEQ ID NO: 30]

5′GACAGCCGGAACACGGCGGCATCAGAGCAGCCGATTGTCTGTTGTGCC
CAGTCATAGCCG 3′
CNeo(+)

[SEQ ID NO: 31]

5′AGACAATCGGCTGCTCTGATGCCGCTTTTgcggcaucagAGCAGccga
uugucuGCGCGTTTTCGCGC 3′

(bold ‘C’ nucleotides represent 5-methyl cytosine. The mismatch nucleotide is underlined. Lower case, 2′-O-methyl RNA residues).

### Analysis of Transformants

Single G418 resistant clones are amplified and DNA is isolated from each individual clone using the Qiagen DNeasy Kit. 100 ng of total DNA is then transformed to E. coli and carbenicillin resistant colonies are selected. Plasmid DNA is isolated from these and conversion of codon 31 is confirmed by sequencing.

### Example 6

### TNE in Human Cells

### Repair of the S205X IL2Rγ Mutation in Human Cell Lines

Cells from a X-linked SCID patient carrying the S201X mutation in exon 5 of the IL2Rγ gene are transfected at an efficiency of ˜80% using Lipofectamine 2000 reagent (Invitrogen) in Opti-MEM I reduced serum medium according to the manufacturers protocol. Cells are transfected with 1 μM of the oligonucleotide HSCIDS201. This is designed to convert the mutation at codon 201 (TGA to TCA) back to the functional codon encoding serine.

HSCIDS201

[SEQ ID NO: 32]

5′GCCCATCCACACTAGGCAAGGAGAACTTATGTCTATAATCCACTGATT
GTTCCTTGAGGAGAAAGAGGATGAGGGAAAGT 3′

(bold ‘C’ nucleotides represent 5-methyl cytosine. The mismatch nucleotide is underlined)

### Proof of Gene Correction at the IL2Rγ S201 Codon

To further characterize clones that undergo conversion at the S201 codon limiting dilution as performed to isolate individual clones. Genomic DNA is isolated from each individual clone using the Qiagen DNeasy Kit. Gene correction is then examined for each clone by amplification of exon 5 of IL2Rγ using the following primers (CAGTGTGGCTTGAGTAGTCA [SEQ ID NO:33] & TAGATCCAGCTGGTTCCAAA [SEQ ID NO:34] that amplify a 613 bp fragment. Gene correction is then confirmed by sequencing the PCR fragments.

Table of sequences:

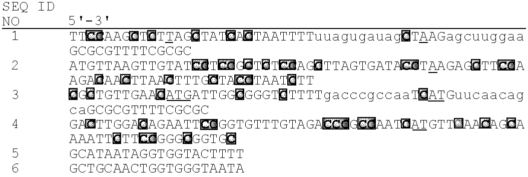

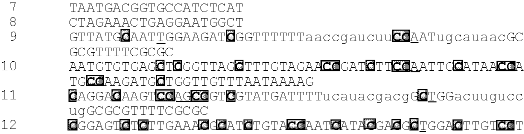

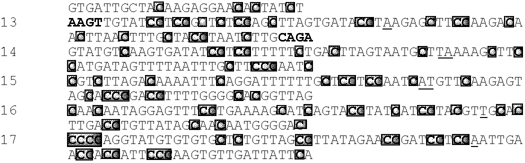

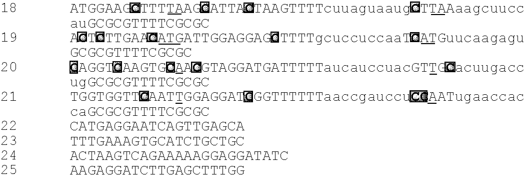

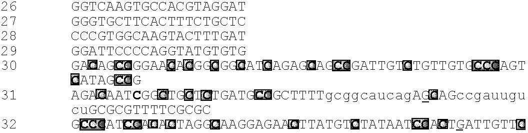

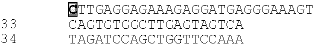

Uppercase: DNA; Uppercase Bold: phosphorothioate linkage; Highlighted bold C: 5-methyl cytosine; Lowercase: 2′O-methyl RNA residues; Underlined: Mismatch nucleotides.

1. An oligonucleotide for targeted alteration of a duplex DNA sequence, the duplex DNA sequence containing a first DNA sequence and a second DNA sequence which is the complement of the first DNA sequence, the oligonucleotide comprising a domain that is capable of hybridizing to the first DNA sequence, which domain comprises at least one mismatch with respect to the first DNA sequence, and wherein the oligonucleotide comprises a section that has a higher degree of methylation than the corresponding part of the second DNA sequence and/or wherein the second DNA has a lower degree of methylation than the corresponding part of the section.

2. Oligonucleotide according to claim 1, wherein the oligonucleotide comprises a section that contains at least one, preferably at least 2, more preferably at least 3 etc., methylated nucleotides more than the corresponding part of the second strand and/or wherein the second strand contains at least one, preferably at least 2, more preferably at least 3 etc., methylated nucleotides less than the corresponding part of the section.

3. Oligonucleotide according to claim 1, which is methylated and wherein the ratio of methylation of the oligonucleotide to the corresponding section on the duplex MDF (Donor)/MDF (Acceptor)>1.0.

4. Oligonucleotide according to claim 1, wherein the nucleotide at the position of the mismatch is not methylated.

5. Oligonucleotide according to claim 1, wherein methylation is preferably adjacent to the mismatch, preferably within 2, 3, 4, 6, 7 nucleotides of the mismatch.

6. Oligonucleotide according to claim 1, having a length from 10 to 500 nucleotides.

7. Oligonucleotide according to claim 1, wherein the (de-)methylated section is the domain.

8. Method for targeted alteration of a duplex acceptor DNA sequence, comprising combining the duplex acceptor DNA sequence with a donor oligonucleotide, wherein the duplex acceptor DNA sequence contains a first DNA sequence and a second DNA sequence which is the complement of the first DNA sequence and wherein the donor oligonucleotide comprises a domain that comprises at least one mismatch with respect to the duplex acceptor DNA sequence to be altered, preferably with respect to the first DNA sequence, and wherein a section of the donor oligonucleotide is methylated to a higher degree than the second DNA sequence and/or wherein the second DNA is demethylated to a lower degree of methylation than the corresponding section of the donor oligonucleotide, in the presence of proteins that are capable of targeted nucleotide exchange.

9. Method according to claim 8, wherein the degree of methylation of the donor oligonucleotide is influenced by chemical means and/or by mutation.

10. Method according to claim 8, wherein de-methylation of one or both strands of the duplex acceptor DNA sequence is by mutation and/or chemical means.

11. Method according to claim 8, wherein the target DNA is from fungi, bacteria, plants, mammals or humans.

12. Method according to claims 8, wherein the duplex DNA is from genomic DNA, linear DNA, artificial chromosomes, nuclear chromosomal DNA, organelle chromosomal DNA, BACs, YACs.

13. Method according to claims 8 for altering a cell, correcting a mutation by restoration to wild type, inducing a mutation, inactivating an enzyme by disruption of coding region, modifying bioactivity of an enzyme by altering coding region, modifying a protein by disrupting the coding region.

14. (canceled)

15. Kit comprising an oligonucleotide as defined in claim 1.

16. Modified genetic material produced by the method of claim 8.

17. Cell comprising the modified genetic material of claim 16.

18. Plant or plant part produced by the method of claim 8.
